(19) United States
(12) Patent Application Publication
(10) Pub. No.: US 2026/0123843 A1
(43) Pub. Date: May 7, 2026

(54) BLOOD PRESSURE PREDICTION SYSTEM AND OPERATING METHOD OF BLOOD PRESSURE PREDICTION SYSTEM

(71) Applicant: ELECTRONICS AND TELECOMMUNICATIONS RESEARCH INSTITUTE, Daejeon (KR)

(72) Inventors: Chan Hwa HONG, Daejeon (KR); Hye Jin KIM, Daejeon (KR)

(21) Appl. No.: 19/297,479

(22) Filed: Aug. 12, 2025

(30) Foreign Application Priority Data
Nov. 5, 2024 (KR) 10-2024-0155153
Feb. 26, 2025 (KR) 10-2025-0025276

(51) Int. Cl. A61B 5/021; A61B 5/00; A61B 5/11; A61B 5/28; A61B 5/352

(52) U.S. Cl. CPC A61B 5/021; A61B 5/1102; A61B 5/28; A61B 5/352; A61B 5/6823; A61B 5/7405; A61B 5/742; A61B 2560/0468

(57) ABSTRACT

A blood pressure prediction system includes a sensing device including a plurality of first electrodes for measuring an electrocardiogram (ECG) signal from a user and a second electrode for measuring a ballistocardiogram (BCG) signal from the user, and a blood pressure prediction apparatus that generates prediction data on a blood pressure of the user based on the ECG and BCG signals. The blood pressure prediction apparatus includes a communication device that receives the ECG and BCG signals from the sensing device, and a processor that acquires first and second sampling data based on the ECG and BCG signals, detects an R peak and a J peak from the first and second sampling data, calculates interval data based on the R and J peaks, and generates prediction data based on a blood pressure prediction equation and the interval data.

### CROSS-REFERENCE TO RELATED APPLICATIONS

This application claims priority under 35 U.S.C. § 119 to Korean Patent Applications No. 10-2024-0155153 filed on Nov. 5, 2024, and No. 10-2025-0025276 filed on Feb. 26, 2025, in the Korean Intellectual Property Office, the disclosures of which are incorporated by reference herein in their entireties.

### BACKGROUND

### 1. Field of the Invention

Embodiments of the present disclosure herein relate to physiological signal processing technology, and more particularly, relate to a blood pressure prediction system based on physiological signal processing and operating method of the blood pressure prediction system.

### 2. Description of Related Art

Blood pressure is an important indicator of cardiovascular health and is essential for early detection and management of chronic diseases such as hypertension. Currently, blood pressure measurement mostly relies on a cuff-type blood pressure gauge. However, this approach makes continuous measurement difficult and can lead to inconvenience in use. Ballistocardiogram (BCG) and Electrocardiogram (ECG) are non-invasive methods of measuring cardiac activity based on minute movements of the heart and electrical signals, respectively, and the time difference between the main peaks (R peak of ECG and J peak of BCG) in the two signals is correlated with blood pressure. Conventional peak detection has been performed by acquiring data for a certain period of time and then predicting blood pressure through analysis of the stored data. This approach makes real-time blood pressure prediction difficult and requires calculating time differences for a large number of R-J values, resulting in time-consuming analysis.

### SUMMARY

Embodiments of the present disclosure provide a blood pressure prediction system and an operating method of the blood pressure prediction system that predict blood pressure by precisely measuring the time difference between the R peak of the ECG signal and the J peak of the BCG signal.

According to an embodiment of the present disclosure, a blood pressure prediction system comprises a sensing device including a plurality of first electrodes for measuring an electrocardiogram (ECG) signal from a user and a second electrode for measuring a ballistocardiogram (BCG) signal from the user, and a blood pressure prediction apparatus that generates prediction data on a blood pressure of the user based on the ECG and BCG signals. The blood pressure prediction apparatus includes a communication device that receives the ECG and BCG signals from the sensing device, and a processor that acquires first sampling data corresponding to the ECG signal and second sampling data corresponding to the BCG signal, detects an R peak and a J peak from the first sampling data and the second sampling data, calculates interval data based on the R peak and the J peak, and generates prediction data based on a blood pressure prediction equation and the interval data.

According to an embodiment of the present disclosure, the processor determines whether the first sampling data and the second sampling data satisfy normal conditions, and detect the R peak and the J peak in response to determining that the normal conditions are satisfied.

According to an embodiment of the present disclosure, the processor determines that the normal conditions are satisfied if a standard deviation of first samples included in the first sampling data falls within a first normal range and a standard deviation of second samples included in the second sampling data falls within a second normal range.

According to an embodiment of the present disclosure, the processor detects, as the R peak, a first target sample among the first samples that has a first maximum value, exceeds a first threshold value, and has a distance from adjacent peaks greater than a minimum peak-to-peak distance, and detects, as the J peak, a second target sample among the second samples that has a second maximum value, exceeds a second threshold value, and has a distance from adjacent peaks greater than the minimum peak-to-peak distance.

According to an embodiment of the present disclosure, the processor determines whether the interval data satisfies a valid condition, and generate the prediction data in response to determining that the valid condition is satisfied.

According to an embodiment of the present disclosure, the processor determines that the valid condition is satisfied if the interval data falls within a valid time range.

According to an embodiment of the present disclosure, the blood pressure prediction apparatus further includes a blood pressure monitor that acquires measurement data for the blood pressure. The processor derives the blood pressure prediction equation based on the measurement data and the interval data.

According to an embodiment of the present disclosure, the processor generates physiological signal data including the prediction data. The blood pressure prediction apparatus further includes a multimedia output device that provides the physiological signal data to the user.

According to an embodiment of the present disclosure, the sensing device is attached in a direction toward the heart with reference to a center of the user's sternum.

According to an embodiment of the present disclosure, the physiological signal data includes at least one of the ECG signal, the BCG signal, and the measurement data.

According to an embodiment of the present disclosure, a method of operating a blood pressure prediction apparatus includes receiving an electrocardiogram (ECG) signal and a ballistocardiogram (BCG) signal from an external sensing device, acquiring first sampling data and second sampling data based on the ECG and BCG signals, determining whether the first sampling data and the second sampling data satisfy normal conditions, detecting an R peak and a J peak from the first sampling data and the second sampling data in response to determining that the normal conditions are satisfied, calculating interval data based on the R peak and the J peak, determining whether the interval data satisfies a valid condition, and generating prediction data on a blood pressure of the user based on a blood pressure prediction equation and the interval data.

According to an embodiment of the present disclosure, the determining of whether the first sampling data and the second sampling data satisfy the normal conditions includes determining whether a standard deviation of first samples included in the first sampling data falls within a first normal range and determining whether a standard deviation of second samples included in the second sampling data falls within a second normal range.

According to an embodiment of the present disclosure, the R peak is a first target sample among the first samples that has a first maximum value, exceeds a first threshold value, and has a distance from adjacent peaks greater than a minimum peak-to-peak distance, and the J peak is a second target sample among the second samples that has a second maximum value, exceeds a second threshold value, and has a distance from adjacent peaks greater than the minimum peak-to-peak distance.

According to an embodiment of the present disclosure, the determining of whether the interval data satisfies the valid condition includes determining whether the interval data falls within a valid time range.

According to an embodiment of the present disclosure, the generating of the prediction data on the blood pressure of the user based on the blood pressure prediction equation and the interval data includes acquiring measurement data for the blood pressure, and generating the blood pressure prediction equation based on the measurement data and the interval data.

According to an embodiment of the present disclosure, the method further includes generating physiological signal data including the prediction data, and outputting the physiological signal data through a multimedia output device.

According to an embodiment of the present disclosure, a blood pressure prediction apparatus includes a communication device that receives an electrocardiogram (ECG) signal and a ballistocardiogram (BCG) signal from an external sensing device, and a processor that acquires first sampling data corresponding to the ECG signal and second sampling data corresponding to the BCG signal, detects an R peak and a J peak from the first sampling data and the second sampling data, calculates interval data based on the R peak and the J peak, and generates prediction data on a blood pressure of the user based on a blood pressure prediction equation and the interval data.

According to an embodiment of the present disclosure, the processor determines whether the first sampling data and the second sampling data satisfy normal conditions, and detects the R peak and the J peak in response to determining that the normal conditions are satisfied.

According to an embodiment of the present disclosure, the processor determines whether the interval data satisfies a valid condition, and generates the prediction data in response to determining that the valid condition is satisfied.

According to an embodiment of the present disclosure, the blood pressure prediction apparatus further includes a blood pressure monitor that acquires measurement data for the blood pressure. The processor generates the blood pressure prediction equation based on the measurement data and the interval data.

### BRIEF DESCRIPTION OF THE DRAWINGS

The above and other objects and features of the present disclosure will become apparent by describing in detail embodiments thereof with reference to the accompanying drawings.

FIG. 1 illustrates a blood pressure prediction system, according to an embodiment of the present disclosure.

FIG. 2 illustrates a sensing element according to an embodiment of the present disclosure.

FIG. 3 illustrates an example of attachment of the sensing device of FIG. 2.

FIG. 4 illustrates an example of a method of operating the sensing device of FIG. 2.

FIG. 5 illustrates a blood pressure prediction apparatus according to an embodiment of the present disclosure.

FIG. 6 illustrates examples of an R peak and a J peak.

FIG. 7 illustrates an example of a method of operating the blood pressure prediction apparatus of FIG. 5.

FIG. 8 illustrates an example of an operating method of deriving a blood pressure prediction equation of the blood pressure prediction apparatus of FIG. 5.

FIG. 9 illustrates an example of physiological signal data.

FIG. 10 illustrates another example of physiological signal data.

### DETAILED DESCRIPTION

Hereinafter, embodiments of the present disclosure may be described in detail and clearly to such an extent that an ordinary one in the art easily implements the present disclosure.

Hereinafter, the preferred embodiments of the present disclosure will be described in detail with reference to the accompanying drawings. The advantages, features, and methods of achieving the present invention will become more apparent from the following detailed description taken in conjunction with the accompanying drawings. However, it should be understood that the present invention is not limited to the embodiments described herein and may be embodied in various other forms. Rather, the embodiments introduced here are provided to make the disclosed content thorough and complete, and to ensure that the concepts of the disclosure are sufficiently conveyed to those skilled in the art, and the disclosure is defined only by the scope of the claims. Throughout the specification, the same reference numerals refer to the same components.

The terms used in the specification are for the purpose of describing the embodiments and are not intended to limit the disclosure. In this specification, the singular form includes the plural form unless specifically stated otherwise in the context. The terms ‘comprise’ and/or ‘comprising’ used in the specification do not exclude the presence or addition of one or more other components, actions, and/or elements. Furthermore, since it is based on preferred embodiments, the reference numerals presented in the description are not necessarily limited by the order of presentation.

The embodiments described in this specification will be explained with reference to ideal examples such as cross-sectional and/or plan views of the disclosure. In the drawings, the thickness of the layers and regions may be exaggerated for the effective explanation of the technical content. Therefore, the shape of the example may be altered due to manufacturing techniques and/or tolerances. Thus, the embodiments of the present disclosure are not limited to the specific forms illustrated, but include changes in the shape created according to the manufacturing process.

Components that are described in the detailed description with reference to the terms “unit”, “module”, “block”, “˜er or ˜or”, etc. and function blocks illustrated in drawings will be implemented with software, hardware, or a combination thereof. For example, the software may be a machine code, firmware, an embedded code, and application software. For example, the hardware may include an electrical circuit, an electronic circuit, a processor, a computer, an integrated circuit, integrated circuit cores, a pressure sensor, an inertial sensor, a microelectromechanical system (MEMS), a passive element, or a combination thereof.

In the present disclosure, each of the phrases such as “A or B,” “at least one of A and B,” “at least one of A or B,” “A, B, or C,” “at least one of A, B, and C,” and “at least one of A, B, or C” is intended to encompass any one of the listed elements and all possible combinations thereof.

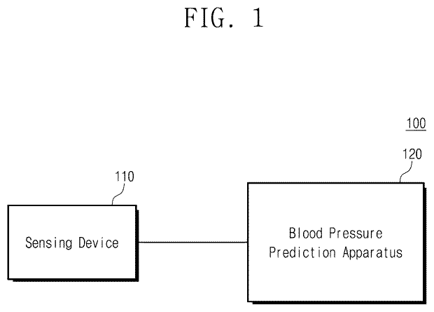

FIG. 1 illustrates a blood pressure prediction system, according to an embodiment of the present disclosure. Referring to FIG. 1, a blood pressure prediction system 100 may include a sensing device 110 and a blood pressure prediction apparatus 120.

The sensing device 110 may include an electrocardiogram (ECG) sensor and a ballistocardiogram (BCG) sensor. The sensing device 110 may measure an ECG signal and a BCG signal from a user by using the ECG sensor and the BCG sensor. The sensing device 110 may transmit the ECG signal and the BCG signal to the blood pressure prediction apparatus 120.

The blood pressure prediction apparatus 120 may predict a blood pressure of the user based on the ECG signal and the BCG signal received from the sensing device 110. The blood pressure prediction apparatus 120 may provide a cardiovascular biological signal to the user in real time based on the predicted blood pressure.

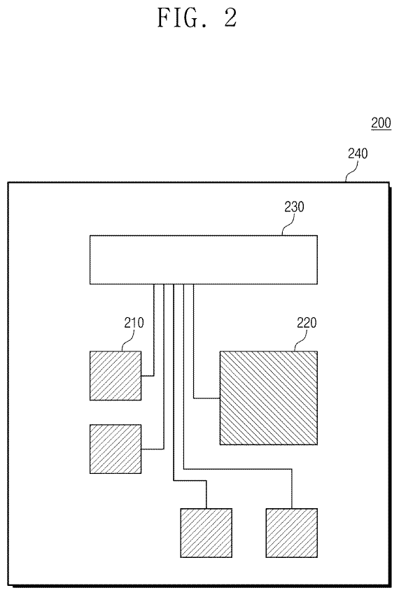

FIG. 2 illustrates a sensing element according to an embodiment of the present disclosure. In FIG. 2, a sensing device 200 may correspond to the sensing device 110 of FIG. 1.

Referring to FIGS. 1 and 2, the sensing device 200 may include a plurality of first electrodes 210, a second electrode 220, a communication device 230, and a substrate 240. The plurality of first electrodes 210, the second electrode 220, and the communication device 230 may be disposed on the substrate 240.

The plurality of first electrodes 210 may measure an ECG signal (or ECG signals) of a user. The ECG signal may indicate electrical activity of the heart. The plurality of electrodes 210 may transmit the ECG signal to the communication device 230.

In an embodiment, the plurality of first electrodes 210 may be disposed at regular intervals from each other. For example, distances between the plurality of first electrodes 210 may be arranged at intervals of 10 mm or more. However, the scope of the present disclosure is not limited thereto.

In an embodiment, the plurality of first electrodes 210 may be disposed in N units (where N is a natural number greater than 1) in each of a first direction D1 and a second direction D2.

The second electrode 220 may measure a BCG signal of the user. The BCG signal may indicate movement (or vibration) of the body caused by aortic ejection based on cardiac contraction. The second electrode 220 may transmit the BCG signal to the communication device 230. In an embodiment, the second electrode 220 may be configured as a sensor thin film utilizing a piezoelectric material.

In an embodiment, the second electrode 220 may be arranged in an array form of N×N formed by the first direction D1 and the second direction D2 on a plane.

The communication device 230 may receive the ECG signal from each of the plurality of first electrodes 210, and may receive the BCG signal from the second electrode 220. In an embodiment, the communication device 230 may be directly connected to the plurality of first electrodes 210 and the second electrode 220 via electrical cables. The communication device 230 may receive the ECG signal and the BCG signal from the first electrodes 210 and the second electrode 220 via electrical cables.

The communication device 230 may transmit the ECG signal and the BCG signal to the blood pressure prediction apparatus 120. In an embodiment, the communication device 230 may communicate with the blood pressure prediction apparatus 120 based on at least one of wireless communication means such as LTE, 5G, Wi-Fi, or the like.

In an embodiment, the plurality of first electrodes 210 and the second electrode 220 may be disposed at regular intervals. For example, the distance between the plurality of first electrodes 210 and the second electrode 220 may be arranged at an interval of 5 mm or more. Thus, interference between the first electrodes 210 and the second electrode 220 may be minimized. However, the scope of the present invention is not limited thereto.

In an embodiment, the substrate 240 may be a flexible substrate (e.g., a flexible printed circuit board (FPCB)). For example, the substrate 240 may be in the form of a stretchable sensor patch.

Although not shown, the sensing device 200 may further include various commercial chips, such as a body temperature sensor and a motion sensor, for physiological signal measurement and life pattern analysis.

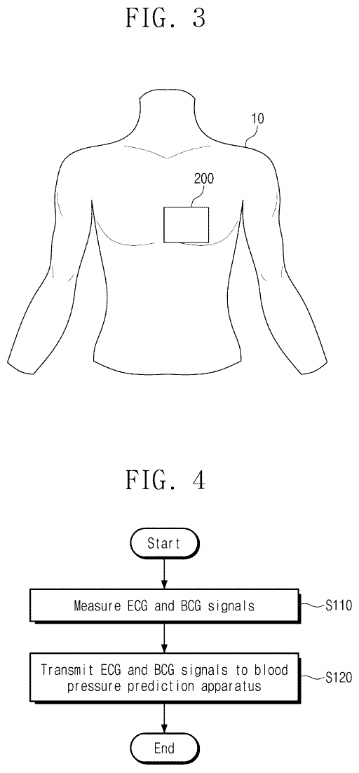

FIG. 3 illustrates an example of attachment of the sensing device of FIG. 2. Referring to FIGS. 2 and 3, the sensing device 200 may be positioned in a region approximately 15 cm away from the sternum, which is the center of the chest of a user 10, in a direction of the heart. Accordingly, the first electrodes 210 may be attached to the sternum region and a left ventricle region of the heart for detecting a P-Q-R-S-T waveform. The first electrodes 210 may measure an ECG signal by using a potential difference between electrodes located in the sternal region and the left ventricle region, thereby enabling accurate signal acquisition. In addition, the second electrode 220 may be disposed at the center of the chest where the pectoralis major muscle is located. When the first electrodes 210 and the second electrode 220 are positioned outside their designated attachment locations, the ECG signal or a BCG signal may not be measured, or may include errors.

FIG. 4 illustrates an example of a method of operating the sensing device of FIG. 2. Referring to FIGS. 1 to 4, in step S110, a sensing device 200 may be attached to the user 10 to measure an ECG signal and a BCG signal.

In step S120, the sensing device 200 may transmit the ECG signal and the BCG signal to the blood pressure prediction apparatus 120 through the communication device 230.

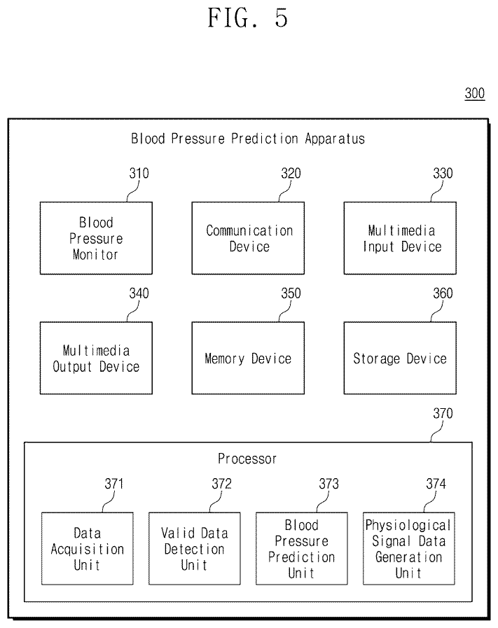

FIG. 5 illustrates a blood pressure prediction apparatus according to an embodiment of the present disclosure. In FIG. 5, a blood pressure prediction apparatus 300 may correspond to the blood pressure prediction apparatus 120 of FIG. 1.

Referring to FIGS. 1 and 5, the blood pressure prediction apparatus 300 may include various electronic devices such as a smartphone, a smart pad, and a wearable device. The blood pressure prediction apparatus 300 may include a blood pressure monitor 310, a communication device 320, a multimedia input device 330, a multimedia output device 340, a memory device 350, a storage device 360, and a processor 370.

The blood pressure monitor 310 may be a photoplethysmography (PPG) sensor-based blood pressure measuring device configured to measure blood pressure a beat-to-beat basis. The blood pressure monitor 310 may measure the blood pressure of a user to derive a blood pressure prediction equation, and may acquire measurement data on the blood pressure of the user.

In an embodiment, the measurement data may include minimum blood pressure information and maximum blood pressure information of the user.

In an embodiment, the blood pressure monitor 310 may be detachable. For example, after the blood pressure prediction equation is derived, the blood pressure monitor 310 may be detached.

The communication device 320 may communicate with the sensing device 110. The communication device 320 may communicate with the sensing device 110 based on at least one of wireless communication means such as LTE, 5G, Wi-Fi, or the like. For example, the communication device 320 may receive an ECG signal and a BCG signal from the sensing device 110.

The multimedia input device 330 may collect information on various actions from the user of the blood pressure prediction apparatus 300. The multimedia input device 330 may include a touch pad, a touch panel, a microphone, or the like.

The multimedia output device 340 may provide the user of the blood pressure prediction apparatus 300 with various types of information, including image information and responses to the actions. The multimedia output device 340 may include a display panel, a speaker, a vibration sensor, a beam projector, or the like.

The memory device 350 may be an operating memory of the blood pressure prediction apparatus 300. The memory device 350 may store instances of an operating system or applications executed by the processor 370, data generated from the processor 370, or user data generated by the user. The memory device 350 may include at least one of various random-access memories, such as a dynamic random-access memory (DRAM), a phase-change RAM (PRAM), a magnetic RAM (MRAM), a ferroelectric RAM (FRAM), a resistive RAM (RRAM), or the like.

The storage device 360 may store source codes of the operating system and applications executing on the blood pressure prediction apparatus 300, data generated from the processor 370, and user data generated by the user. The storage device 360 may include at least one of a variety of non-volatile memory devices, such as flash memory devices, PRAM, MRAM, FRAM, RRAM, or the like.

The processor 370 may execute the operating system for controlling the blood pressure prediction apparatus 300, and execute the various applications. The processor 370 may include at least one of various processing cores including a central processing unit (CPU), a graphics processing unit (GPU), a neural processor, a digital signal processor, an image signal processor, and the like.

The processor 370 may generate prediction data on the blood pressure of the user based on the ECG signal and the BCG signal received from the sensing device 110. For example, the processor 370 may generate the prediction data on the blood pressure of the user based on the ECG signal and the BCG signal by using a blood pressure prediction algorithm. The processor 370 may generate physiological signal data based on the prediction data.

The processor 370 may include a data acquisition unit 371, a valid data detection unit 372, a blood pressure prediction unit 373, and a physiological signal data generation unit 374.

The data acquisition unit 371 may collect data usable for detecting an R peak and a J peak, based on the ECG signal and the BCG signal. For example, the data acquisition unit 371 may sample the ECG signal to acquire first sampling data including first samples, and sample the BCG signal to acquire second sampling data including second samples.

In an embodiment, the data acquisition unit 371 may sample the ECG signal and the BCG signal at a frequency of 1 kHz or higher, respectively. For example, the data acquisition unit 371 may acquire the first sampling data including 1000 first samples based on the ECG signal. Similarly, the data acquisition unit 371 may acquire the second sampling data including 1000 second samples based on the BCG signal. However, the scope of the present disclosure is not limited thereto, and the data acquisition unit 371 may sample the ECG signal and the BCG signal, respectively, at any frequency.

The data acquisition unit 371 may determine whether the acquired first sampling data and the acquired second sampling data satisfy normal conditions. For example, the data acquisition unit 371 may determine whether a standard deviation of the first samples included in the first sampling data falls within a first normal range, and determine whether a standard deviation of the second samples included in the second sampling data falls within a second normal range. If the standard deviation of the first samples falls within the first normal range and the standard deviation of the second samples falls within the second normal range, the data acquisition unit 371 may determine that the first sampling data and the second sampling data satisfy the normal conditions.

In an embodiment, the first normal range may be between 50 and 300, and the second normal range may be between 50 and 1000. However, the scope of the present disclosure is not limited thereto.

The valid data detection unit 372 may detect the R peak and the J peak based on the first sampling data and the second sampling data. For example, in response to determining that the first sampling data and the second sampling data satisfy the normal conditions, the valid data detection unit 372 may detect the R peak based on the first sampling data, and detect the J peak based on the second sampling data.

The valid data detection unit 372 can detect the R peak from the first sampling data and detect the J peak from the second sampling data by using a peak detection algorithm.

For example, the valid data detection unit 372 may detect the R peak among the first samples included in the first sampling data based on Equations 1 to 7 below.

$$x [ n ] > x [ n - 1 ] \qquad [ Equation 1 ]$$

$$x [ n ] > x [ n + 1 ] \qquad [ Equation 2 ]$$

$${x}^{′} [ n ] = x [ n ] - x [ n - 1 ] \qquad [ Equation 3 ]$$

$${x}^{′} [ n ] < 0 \qquad [ Equation 4 ]$$

$${x}^{′} [ n - 1 ] > 0 \qquad [ Equation 5 ]$$

$$x [ n ] > T \qquad [ Equation 6 ]$$

$$\vert {n}_{i} - {n}_{j} \vert \geq D \qquad [ Equation 7 ]$$

In Equations 1 through 7, x[n] may represent a value of an n-th sample, x[n−1] may represent a value of an (n−1)-th sample, x[n+1] may represent a value of an (n+1)-th sample, x′[n] may represent a difference value of the n-th sample, x′[n−1] may represent a difference value of the (n−1)-th sample, T may represent a threshold value, nᵢ may represent an i-th peak, nⱼ may represent a j-th peak, and nᵢ and nⱼ may represent consecutive peaks, |nᵢ−nⱼ| may represent a distance between the i-th and j-th peaks, and D may represent a minimum peak-to-peak distance.

The valid data detection unit 372 may detect a sample that satisfies Equations 1 to 7 among the first samples as the R peak.

On the other hand, Equations 1 to 5 may represent definitions of peaks in a discrete signal. Therefore, the valid data detection unit 372 may detect, as the R peak, a sample (e.g., a first target sample) among the first samples that satisfies a definition of a peak, has a value greater than a first threshold value, and has a distance from adjacent peaks greater than the minimum peak-to-peak distance. Alternatively, the valid data detection unit 372 may detect, as the R peak, a sample (e.g., the first target sample) among the first samples that has a maximum value (e.g., a first maximum value), has a value greater than the first threshold value, and has a distance from the adjacent peaks greater than the minimum peak-to-peak distance.

Similarly, the valid data detection unit 372 may detect, as the J peak, a sample (e.g., a second target sample) among the second samples that satisfies the definition of the peak, has a value greater than a second threshold value, and has a distance from adjacent peaks greater than the minimum peak-to-peak distance. Alternatively, the valid data detection unit 372 may detect, as the J peak, a sample (e.g., the second target sample) that has a maximum value, a value greater than the second threshold value, and a distance from adjacent peaks greater than the minimum peak-to-peak distance.

In an embodiment, each of the first threshold value and the second threshold value may be determined based on a sample having a maximum value. That is, the first threshold value and the second threshold value may be different from each other. For example, the first threshold value may be 95% of a value of a sample having a maximum value among the first samples, and the second threshold value may be 95% of a value of a sample having a maximum value among the second samples.

The valid data detection unit 372 may calculate interval data indicating a time difference (or a time interval) between the R peak and the J peak based on the detected R peak and the detected J peak. For example, the valid data detection unit 372 may calculate the interval data by subtracting a time point of the R peak and a time point of the J peak.

The valid data detection unit 372 may determine whether the interval data satisfy a valid condition. For example, the valid data detection unit 372 may determine whether the interval data falls within a valid time range. If the interval data falls within the valid time range, the valid data detection unit 372 may determine that the interval data satisfies the valid condition.

In an embodiment, the valid time range may be between 0.02 seconds and 0.1 seconds. However, since there are individual differences depending on the user, the valid time range may vary.

The blood pressure prediction unit 373 may generate prediction data on the blood pressure of the user based on the blood pressure prediction equation and the interval data. For example, in response to determining that the interval data satisfies the valid condition, the blood pressure prediction unit 373 may derive the blood pressure prediction equation based on the measurement data acquired from the blood pressure monitor 310 and the interval data. The blood pressure prediction equation may be an equation indicating a correlation between the blood pressure and the interval data.

Specifically, the blood pressure prediction unit 373 may derive the blood pressure prediction equation in the form of a linear equation such as “y=a*x+b” by using a linear regression equation. In this case, x, which is an independent variable, may indicate a time difference between the R peak and the J peak, and y, which is a dependent variable, may indicate blood pressure.

The blood pressure prediction unit 373 may generate the prediction data on the blood pressure of the user by substituting the interval data as the independent variable of the derived blood pressure prediction equation.

In an embodiment, when a value of the interval data is small, a value of the prediction data may be high, and when the value of the interval data is large, the value of the prediction data may be small.

The physiological signal data generation unit 374 may generate physiological signal data related to a cardiovascular system based on the generated prediction data. For example, the physiological signal data generation unit 374 may generate the physiological signal data including the prediction data.

In an embodiment, the physiological signal data may further include at least one of the ECG signal, the BCG signal, or measurement data acquired from the blood pressure monitor 310.

When the physiological signal data is generated, the blood pressure prediction apparatus 300 may provide the physiological signal data to the user through the multimedia output device 340. For example, the processor 370 may control the multimedia output device 340 to output the physiological signal data via the GUI.

As described above, the blood pressure prediction apparatus 300 may predict the blood pressure of the user and provide the predicted blood pressure to the user in real time without a delay time.

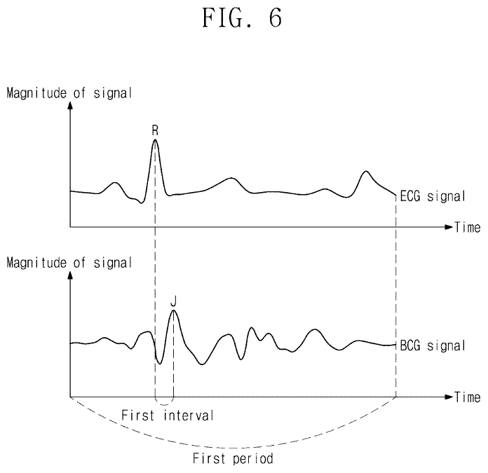

FIG. 6 illustrates examples of an R peak and a J peak. Referring to FIGS. 5 and 6, an R peak may correspond to an ECG signal having a maximum value during a first period, and a J peak may correspond to a BCG signal having a maximum value for the first period. The blood pressure prediction apparatus 300 may acquire first sampling data and second sampling data based on the ECG signal and the BCG signal, detect the R peak and the J peak based on the first sampling data and the second sampling data, and calculate interval data corresponding to a first interval.

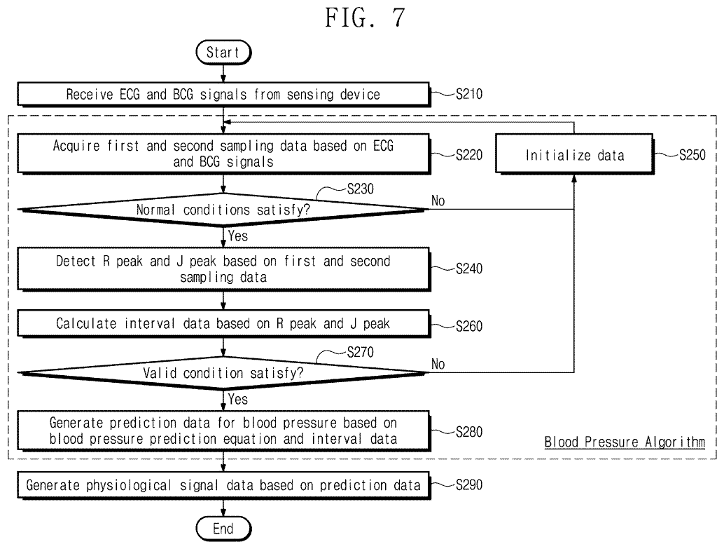

FIG. 7 illustrates an example of a method of operating the blood pressure prediction apparatus of FIG. 5. Referring to FIGS. 1, 5, and 7, in step S210, a blood pressure prediction apparatus 300 may receive an ECG signal and a BCG signal from a sensing device 110. For example, the blood pressure prediction apparatus 300 may receive the ECG signal and a BCG signal from the sensing device 110 through a communication device 320.

In step S220, the blood pressure prediction apparatus 300 may acquire first sampling data and second sampling data based on the ECG signal and the BCG signal. For example, the blood pressure prediction apparatus 300 may sample the ECG signal to acquire the first sampling data including first samples. Likewise, the blood pressure prediction apparatus 300 may sample the BCG signal to acquire the second sampling data including second samples.

In an embodiment, the blood pressure prediction apparatus 300 may store the first sampling data and the second sampling data in at least one of a memory device 350 or a storage device 360.

In step S230, the blood pressure prediction apparatus 300 may determine whether the first sampling data and the second sampling data satisfy normal conditions.

For example, the blood pressure prediction apparatus 300 may determine whether a standard deviation of the first samples included in the first sampling data falls within a first normal range, and determine whether a standard deviation of the second samples included in the second sampling data falls within a second normal range.

If the standard deviation of the first samples falls within the first normal range and the standard deviation of the second samples falls within the second normal range, the blood pressure prediction apparatus 300 may determine that the first sampling data and the second sampling data satisfy the normal conditions. If the standard deviation of the first samples is outside the first normal range, the standard deviation of the second samples is outside the second normal range, or the standard deviations of the first samples and the second samples are outside the first range and the second normal range, respectively, the blood pressure prediction apparatus 300 may determine that the first sampling data and the second sampling data do not satisfy the normal conditions.

If it is determined that the first sampling data and the second sampling data satisfy the normal conditions, in step S240, the blood pressure prediction apparatus 300 may detect an R peak and a J peak based on the first sampling data and the second sampling data. For example, the blood pressure prediction apparatus 300 may detect the R peak based on the first sampling data and detect the J peak based on the second sampling data by using a peak detection algorithm.

If it is determined that the first sampling data and the second sampling data do not satisfy the normal conditions, in step S250, the blood pressure prediction apparatus 300 may initialize the data (e.g., the first sampling data and the second sampling data). In an embodiment, if the first sampling data and the second sampling data are stored in at least one of a memory device 350 or a storage device 360, the blood pressure prediction apparatus 300 may erase the stored data. After initializing the data, the blood pressure prediction apparatus 300 may perform step S220 again.

In step S260, the blood pressure prediction apparatus 300 may calculate interval data based on the R peak and the J peak. For example, the blood pressure prediction apparatus 300 may calculate the interval data by subtracting a time point of the R peak and a time point of the J peak.

In an embodiment, the blood pressure prediction apparatus 300 may store the interval data in at least one of the memory device 350 or the storage device 360.

In step S270, the blood pressure prediction apparatus 300 may determine whether the interval data satisfies a valid condition. For example, the blood pressure prediction apparatus 300 may determine whether the interval data falls within a valid time range.

If the interval data falls within the valid time range, the blood pressure prediction apparatus 300 may determine that the interval data satisfies the valid condition. If the interval data is outside the valid time range, the blood pressure prediction apparatus 300 may determine that the interval data does not satisfy the valid condition.

If it is determined that the interval data satisfies the valid condition, in step S280, the blood pressure prediction apparatus 300 may generate prediction data on a blood pressure of a user based on blood pressure prediction equation and the interval data. The blood pressure prediction equation may be an equation indicating a correlation between the blood pressure and the interval data. In an embodiment, the blood pressure prediction apparatus 300 may store the prediction data in at least one of the memory device 350 or the storage device 360.

If it is determined that the interval data does not satisfy the valid condition, the blood pressure prediction apparatus 300 may perform step S250 again. In an embodiment, when at least one of the first sampling data, the second sampling data, and the interval data is stored in at least one of the memory device 350 or the storage device 360, the blood pressure prediction apparatus 300 may erase the stored data.

In step S290, the blood pressure prediction apparatus 300 may generate physiological signal data associated with the cardiovascular system based on the prediction data. In an embodiment, the blood pressure prediction apparatus 300 may store the physiological signal data in at least one of the memory device 350 or the storage device 360.

In an embodiment, the blood pressure prediction apparatus 300 may provide the physiological signal data to the user via a multimedia output device 340.

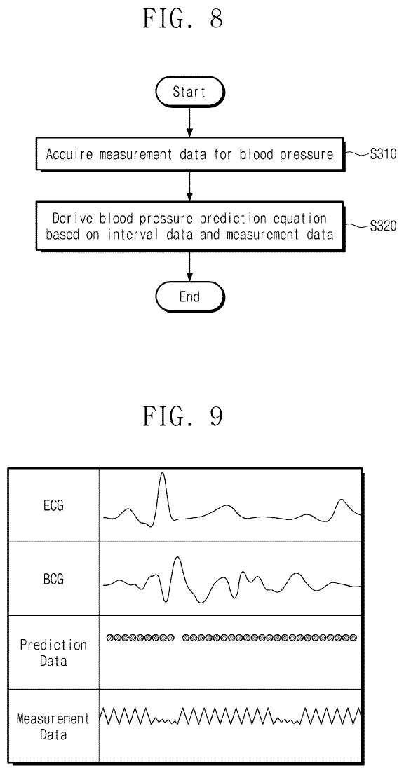

FIG. 8 illustrates an example of an operating method of deriving a blood pressure prediction equation of the blood pressure prediction apparatus of FIG. 5. In FIG. 8, steps S310 and S320 may be included in step S280 of FIG. 7.

Referring to FIGS. 5, 7, and 8, in step S310, a blood pressure prediction apparatus 300 may acquire measurement data on a blood pressure of a user. For example, the blood pressure monitor 310 of the blood pressure predicting device 300 may measure the blood pressure of the user, and acquire the measurement data on the blood pressure of the user.

In step S320, the blood pressure prediction apparatus 300 may derive a blood pressure prediction equation based on interval data and the measurement data. In an embodiment, the blood pressure prediction apparatus 300 may store the blood pressure prediction equation in at least one of the memory device 350 or the storage device 360.

FIG. 9 illustrates an example of physiological signal data. Referring to FIGS. 2, 5, and 9, a blood pressure prediction apparatus 300 may output physiological signal data through a multimedia output device 340. The physiological signal data may then be represented in the form of a graph. The physiological signal data may include an ECG signal, a BCG signal, prediction data, and the measurement data.

In an embodiment, if a sensing device 200 includes various sensors such as a body temperature sensor and a motion sensor, the physiological signal data may include signals or data acquired from various sensors.

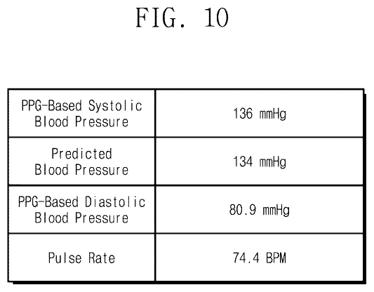

FIG. 10 illustrates another example of physiological signal data. Referring to FIGS. 2, 5, and 10, a blood pressure prediction apparatus 300 may output physiological signal data through a multimedia output device 340. The physiological signal data may then be represented in the form of a table. The physiological signal data may include information about PPG-based maximum blood pressure (e.g., PPG-based systolic blood pressure), predicted blood pressure, PPG-based minimum blood pressure (e.g., PPG-based diastolic blood pressure), and pulse rate.

In an embodiment, if a sensing device 200 includes various sensors such as a body temperature sensor and a motion sensor, the physiological signal data may include signals or data acquired from various sensors.

In the above embodiments, components according to the present disclosure are described by using the terms “first”, “second”, “third”, and the like. However, the terms “first”, “second”, “third”, and the like may be used to distinguish components from each other and do not limit the present disclosure. For example, the terms “first”, “second”, “third”, and the like do not involve an order or a numerical meaning of any form.

The above descriptions are detail embodiments for carrying out the present disclosure. Embodiments in which a design is changed simply or which are easily changed may be included in the present disclosure as well as an embodiment described above. In addition, technologies that are easily changed and implemented by using the above embodiments may be included in the present disclosure.

According to the present disclosure, a blood pressure prediction system may predict blood pressure non-invasively without the need for a cuff, and it may predict the blood pressure in real time without the need for separate analysis time. Additionally, the blood pressure prediction system may improve the accuracy of peak detection through standard deviation-based data validation and threshold value setting.

According to the present disclosure, the blood pressure prediction system may easily monitor the blood pressure not only in medical environments but also at home, and may provide blood pressure prediction services to various user groups through the application of user-specific correlations.

What is claimed is:

1. A blood pressure prediction system comprising:
a sensing device including a plurality of first electrodes for measuring an electrocardiogram (ECG) signal from a user and a second electrode for measuring a ballistocardiogram (BCG) signal from the user; and
a blood pressure prediction apparatus configured to generate prediction data on a blood pressure of the user based on the ECG and BCG signals,
wherein the blood pressure prediction apparatus includes:
a communication device configured to receive the ECG and BCG signals from the sensing device; and
a processor configured to acquire first sampling data corresponding to the ECG signal and second sampling data corresponding to the BCG signal, detect an R peak and a J peak from the first sampling data and the second sampling data, calculate interval data based on the R peak and the J peak, and generate the prediction data based on a blood pressure prediction equation and the interval data.

2. The blood pressure prediction system of claim 1, wherein the processor configured to:
determine whether the first sampling data and the second sampling data satisfy normal conditions; and
detect the R peak and the J peak in response to determining that the normal conditions are satisfied.

3. The blood pressure prediction system of claim 2, wherein the processor determines that the normal conditions are satisfied if a standard deviation of first samples included in the first sampling data falls within a first normal range and a standard deviation of second samples included in the second sampling data falls within a second normal range.

4. The blood pressure prediction system of claim 3, wherein the processor configured to:
detect, as the R peak, a first target sample among the first samples that has a first maximum value, exceeds a first threshold value, and has a distance from adjacent peaks greater than a minimum peak-to-peak distance, and
detect, as the J peak, a second target sample among the second samples that has a second maximum value, exceeds a second threshold value, and has a distance from adjacent peaks greater than the minimum peak-to-peak distance.

5. The blood pressure prediction system of claim 4, wherein the processor configured to:
determine whether the interval data satisfies a valid condition; and
generate the prediction data in response to determining that the valid condition is satisfied.

6. The blood pressure prediction system of claim 5, wherein the processor determines that the valid condition is satisfied if the interval data falls within a valid time range.

7. The blood pressure prediction system of claim 6, further comprising a blood pressure monitor configured to acquire measurement data for the blood pressure,
wherein the processor derives the blood pressure prediction equation based on the measurement data and the interval data.

8. The blood pressure prediction system of claim 7, wherein the processor generates physiological signal data including the prediction data, and
wherein the blood pressure prediction apparatus further includes a multimedia output device configured to provide the physiological signal data to the user.

9. The blood pressure prediction system of claim 8, wherein the sensing device is attached in a direction toward the heart with reference to a center of the user's sternum.

10. The blood pressure prediction system of claim 8, wherein the physiological signal data includes at least one of the ECG signal, the BCG signal, or the measurement data.

11. A method of operating a blood pressure prediction apparatus, the method comprising:
receiving an electrocardiogram (ECG) signal and a ballistocardiogram (BCG) signal from an external sensing device;
acquiring first sampling data and second sampling data based on the ECG and BCG signals, respectively;
determining whether the first sampling data and the second sampling data satisfy normal conditions;
detecting an R peak and a J peak from the first sampling data and the second sampling data in response to determining that the normal conditions are satisfied;
calculating interval data based on the R peak and the J peak;
determining whether the interval data satisfies a valid condition; and
generating prediction data on a blood pressure of the user based on a blood pressure prediction equation and the interval data.

12. The method of claim 11, wherein the determining of whether the first sampling data and the second sampling data satisfy the normal conditions includes:
determining whether a standard deviation of first samples included in the first sampling data falls within a first normal range; and
determining whether a standard deviation of second samples included in the second sampling data falls within a second normal range.

13. The method of claim 12,
wherein the R peak is a first target sample among the first samples that has a first maximum value, exceeds a first threshold value, and has a distance from adjacent peaks greater than a minimum peak-to-peak distance, and
wherein the J peak is a second target sample among the second samples that has a second maximum value, exceeds a second threshold value, and has a distance from adjacent peaks greater than the minimum peak-to-peak distance.

14. The method of claim 13, wherein determining of whether the interval data satisfies the valid condition includes determining whether the interval data falls within a valid time range.

15. The method of claim 14, wherein the generating of the prediction data on the blood pressure of the user based on the blood pressure prediction equation and the interval data includes:
acquiring measurement data for the blood pressure; and
generating the blood pressure prediction equation based on the measurement data and the interval data.

16. The method of claim 15, further comprising:
generating physiological signal data including the prediction data; and
outputting the physiological signal data through a multimedia output device.

17. A blood pressure prediction apparatus comprising:
a communication device configured to receive an electrocardiogram (ECG) signal and a ballistocardiogram (BCG) signal of a user from an external sensing device; and
a processor configured to acquire first sampling data corresponding to the ECG signal and second sampling data corresponding to the BCG signal, detect an R peak and a J peak from the first sampling data and the second sampling data, calculate interval data based on the R peak and the J peak, and generate prediction data on a blood pressure of the user based on a blood pressure prediction equation and the interval data.

18. The blood pressure prediction apparatus of claim 17, wherein the processor configured to:
determine whether the first sampling data and second sampling data satisfy normal conditions; and
detect the R peak and the J peak in response to determining that the normal conditions are satisfied.

19. The blood pressure prediction apparatus of claim 18, wherein the processor configured to:
determine whether the interval data satisfies a valid condition: and
generate the prediction data in response to determining that the valid condition is satisfied.

20. The blood pressure prediction apparatus of claim 19, further comprising a blood pressure monitor configured to acquire measurement data for the blood pressure,
wherein the processor generates the blood pressure prediction equation based on the measurement data and the interval data.
